(19) United States
(12) Patent Application Publication
(10) Pub. No.: US 2002/0106396 A1
(43) Pub. Date: Aug. 8, 2002

(54) Food product

(76) Inventors: Leslie John Herzog, Nyack, NY (US); Sanitha Shamanna Bhima Rao, Parsipanny, NJ (US); Matthew Richard Park, River Edge, NJ (US); John David Crump, Tenafly, NJ (US); Charles Bee Brown, Rutherford, NJ (US); Podutoori Ravinder Reddy, Columbia, MD (US); Mandy Kim Chen, Baltimore, MD (US); Matthew Patrick, Annapolis, MD (US); Michael Budd, Lincoln Park, NJ (US)

Correspondence Address: UNILEVER, PATENT DEPARTMENT, 45 RIVER ROAD, EDGEWATER, NJ, 07020, US

(21) Appl. No.: 09/563,569

(22) Filed: May 3, 2000

Related U.S. Application Data
(60) Provisional application No. 60/132,659, filed on May 5, 1999.

(51) Int. Cl. A61K 47/00

(52) U.S. Cl. 424/439

(57) ABSTRACT

Food products containing isoflavones and calcium are taught where the weight ratio of isoflavones to calcium is from 1 to 2 to 1 to 50.

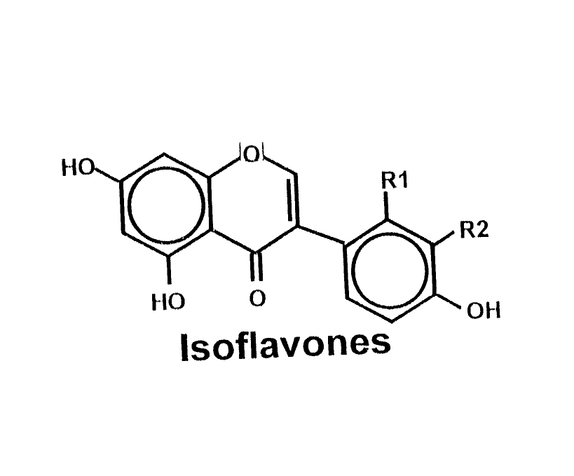

### TECHNICAL FIELD OF THE INVENTION

The invention relates to food products and methods of preparation of food products. In particular to food products containing one or more isoflavone compounds.

### BACKGROUND TO THE INVENTION

Isoflavones have been proposed as ingredients for food and/or pharmaceutical products.

For example WO 98/08503 (Novogen Research) mentions the use of certain isoflavone compounds in therapeutic uses, methods, compounds, formulations, and drinks and foodstuffs.

WO 96/10341 (Schouten Industries) discloses food or health products comprising substantially pure hypocotyls of the seeds of Glycine max

WO 93/23069 (Kelly) discloses health supplements comprising a health supplementary amount of a phyto-estrogen selected from genistein, daidzein, biochanin A, and/or formononetin.

Despite the fact that isoflavones have been suggested to have a positive effect on various health effects, the actual marketing of food products supplemented with isoflavones has up till now not taken place on a large scale.

One of the problems in formulating food products with isoflavones is that it is often difficult to find product formulations which on the one hand provide the desired health benefits and on the other hand can fairly easily be formulated at a reasonable cost. A further problem in the formulation of food products with isoflavones is that on the one hand—for cost and taste reasons—a relatively low level of supplemented ingredients is preferred while on the other hand it is desirable to have a level which leads to an appropriate bioavailability in the human body.

Furthermore consumers these days have a clear preference for food products which form part of their common food habits and which, in themselves, have a clear contribution towards a healthy diet by which no longer additives or a special diet needs to be taken.

The present invention aims to provide food products which are part of the common diet and which comprise isoflavones and calcium in amounts sufficient to provide balanced and advantageous health benefits to consumers, especially to middle aged women aged 35 to 65. By this invention, the consumer need not alter their daily consuming habits, while the risk towards various diseases for example osteoporosis and/or menopausal syndrome and/or hot flushes and/or cardiovascular disease and/or breast cancer and/or migraine can be reduced by the intake of the food product.

It has now been found that one or more of the above problems can be solved if a food product is prepared which contains isoflavones in combination with Calcium, whereby the ingredients are used in specific amounts.

### SUMMARY OF INVENTION

Accordingly the present invention relates to a food product comprising isoflavones and calcium, wherein the weight ratio of isoflavones to calcium is from 1 to 2 to 1 to 50.

The invention also relates to a method of preparing a food product whereby one or more isoflavones and one or more calcium salts are included in the product.

Preferably food products of the invention are part of the normal daily diet, for example margarines or other spreads or oil based products, bakery products, dairy products e.g. yoghurt, cheese and milk-based drinks, beverages e.g. soft drinks, fruit juices and tea and coffee based drinks, sauces especially dressings and mayonnaise and confectionery products e.g. frozen confectionery products such as water-ice or ice-cream.

### DETAILED DESCRIPTION OF THE INVENTION

Isoflavones belong to the class of flavonoids. The basic formula of isoflavones is given in the Figure. R1 and R2 may vary independently, but are usually hydrogen or hydroxyl. Examples of isoflavone compounds are given in WO 98/8503.

Preferred isoflavones for use in the present invention have anti-oxidant properties. In this context it is preferred that the isoflavones comprise at least one hydroxy substituent, more preferred 2 to 4 hydroxy groups. Examples of preferred isoflavones are given in formula (1) to (19) of WO 98/8503, especially preferred isoflavones are Daidzein, Genistein, dihydrodaidzein and dihydrogenistein.

Isoflavones can be produced chemically or can be isolated from natural sources e.g. plants. Especially suitable is the isolation of isoflavones from plants for example from linseed, lentils, beans, chickpeas, green peas and soya. Most preferred is to use isoflavones which have been isolated from soya.

Food products according to the invention comprise isoflavones and calcium, wherein the weight ratio of isoflavones to calcium is from 1 to 2 to 1 to 50, more preferred 1 to 3 to 1 to 30, most preferred from 1 to 5 to 1 to 14.

Typical amounts of isoflavones in the food product can suitably be adapted to the desirable intake and the average size of a serving. Preferably the level of isoflavone is chosen such that a normal serving of the food product comprises 1 to 200 milligrams of isoflavone, more preferred 2 to 100 milligrams, especially preferred 4 to 40 milligrams, most preferred 5 to 15 milligrams. Typical sizes of a single serving are given below.

Isoflavones may be added in their glycosylated form (which is often their natural form) or may advantageously be added as an aglycone. For the purpose of the invention the weight of the isoflavone refers to the isoflavone as if in aglycone form.

Typical amounts of calcium in the food product are 2 to 50 times the amount of isoflavone, more preferred 3 to 30 times, most preferred 5 to 14 times. Taking into account this ratio in combination with the desirable intake of calcium and the average size of serving the absolute level of calcium in the food product may be determined. Preferably the level of calcium is chosen such that its weight ratio to the amount of isoflavone is as above and also that a normal serving of the food product comprises 100 to 2000 milligram of calcium, more preferred 110 to 1000 milligram, especially preferred 120 to 500 milligram, most preferred 120 to 200 milligram.

In this context is should be noted that some food products, e.g. dairy products already contain calcium as one of their ingredients, to these products generally calcium will be added to increase the total level into the desired range.

Calcium may be added in any suitable form, but generally an edible salt of calcium will be used. For some products, for example products to prepare tea based beverages, it is especially preferable if a calcium salt is used which is relatively water soluble to allow incorporation into the food product and uptake of the calcium in the human tract. Examples of preferred calcium salts are calcium maleate, calcium tartrate, calcium gluconate, calcium lactate, calcium acetate, especially preferred are calcium lactate and calcium gluconate. For other products, for example margarine or other spreads, it may be advantageous to use calcium salts which may form small crystals in the product for example calcium carbonate or calcium phosphate salts such as calcium tripolyphosphate.

The invention may usefully be applied to a variety of food products. Especially preferred are the use in food products which tend to be part of the daily diet. These products can suitably be used for repeated and regular dosage of the combination of isoflavones and calcium over prolonged periods and therewith provide good health benefits.

Examples of preferred food products are margarines or other spreads or oil based products, bakery products, dairy products e.g. yoghurt, cheese and milk-based drinks, beverages e.g. soft drinks, fruit juices and tea and coffee based drinks, sauces especially dressings and mayonnaise and confectionery products e.g. frozen confectionery products such as water-ice or ice-cream. Especially preferred is the use in food products selected from the group of margarines and other spreads, tea based beverages, dressings and frozen confectionery products.

Food products according to the invention are advantageously recommended for use by women of the age of 35 to 65. A typical recommended diet may involve the administering of 2-100 servings of products of the invention per week, more preferred 5-50 per week. This, of course, depends on the amount of isoflavone and calcium in the product. Especially preferred is the use of one or more products of the invention in amounts such that per week on average 20 to 1000 mg of isoflavones is consumed via products of the invention, more preferred 50 to 500 mg, most preferred 70 to 500 mg.

For margarine or other spreads the number of servings (about 14 grams) advantageously is 5-25 per week, for example 10-20. For frozen confectionery products the number of servings (about 66 grams) advantageously is 2-14, more preferred 3-10. For tea based products (about 250 ml) the number of servings is preferably 5 to 45 per week, more preferred 10 to 30. For dressings or mayonnaise the number of servings (about 30 g) is advantageously 2-40 per week, more preferred 5-20 per week.

The invention will now be further illustrated by the description of suitable embodiments of the preferred food products. It is believed to be well within the ability of the skilled person to use the teaching provided therewith to prepare other products of the invention.

### Margarines and Other Spreads

Typically these are oil in water or water in oil emulsions, also spreads which are substantially fat free are covered. Typically these products are spreadable and not pourable at the temperature of use e.g. 2-10 C. Fat levels may vary in a wide range e.g. full fat margarines with 60-90 wt % of fat, medium fat margarines with 30-60 wt % of fat, low fat products with 10-30 wt % of fat and very low or fat free margarines with 0 to 10 wt % of fat.

The fat in the margarine or other spread may be any edible fat, often used are soybean fat, rapeseed oil, sunflower oil and palm oil. Fats may be used as such or in modified form e.g. hydrogenated, esterified, refined etc. Other suitable oils are well known in the art and may be selected as desired.

Examples of spreads other than margarines are cheese spreads, sweet spreads etc.

Optional further ingredients of spreads may be emulsifiers, colourants, vitamins, preservatives, emulsifiers, gums, thickeners etc. The balance of the product will normally be water.

A typical size for an average serving of margarine or other spreads is 14 grams. Preferred isoflavone levels in the margarine or spread are 0.007 to 1.5 wt %, more preferred 0.015 to 0.7 wt %, especially preferred 0.03 to 0.3 wt %, most preferred 0.035 to 0.1 wt %. Preferred calcium levels are 0.7 to 15 wt %, more preferred 0.8 to 7 wt %, especially preferred 0.85 to 3.5 wt %, most preferred 0.85 to 1.5 wt %.

### Frozen Confectionery Products

For the purpose of the invention the term frozen confectionery product includes milk containing frozen confections such as ice-cream, frozen yoghurt, sherbet, sorbet, ice milk and frozen custard, water-ices, granitas and frozen fruit purees.

Preferably the level of solids in the frozen confection (e.g. sugar, fat, flavouring etc) is more than 3 wt %, more preferred from 10 to 70 wt, for example 40to 70 wt %.

Ice-cream will typically comprise 2 to 20 wt % of fat, 0 to 20 wt % of sweeteners, 2 to 20 wt % of non-fat milk components and optional components such as emulsifiers, stabilisers, preservatives, flavouring ingredients, vitamins, minerals, etc, the balance being water. Typically ice-cream will be aerated e.g. to an overrun of 20 to 400%, more general 40 to 200% and frozen to a temperature of from −2 to −200 C., more general −10 to −30 C. Ice-cream normally comprises calcium at a level of about 0.1 wt %.

A typical size of an average serving of frozen confectionery material is 66 grams. Preferred isoflavone levels in the frozen confectionery are 0.0015 to 0.3 wt %, more preferred 0.003 to 0.15 wt %, especially preferred 0.006 to 0.06 wt %, most preferred 0.008 to 0.025 wt %. Preferred calcium levels are 0.15 to 3 wt %, more preferred 0.17 to 1.5 wt %, especially preferred 0.18 to 0.75 wt %, most preferred 0.18 to 0.3 wt %.

### Tea Based Products

For the purpose of this invention the term tea based products refers to products containing tea or tea replacing herbal compositions e.g. tea-bags, leaf tea, herbal tea bags, herbal infusions, powdered tea, powdered herbal tea, ice-tea, ice herbal tea, carbonated ice tea, carbonated herbal infusions etc.

Typically some tea based products of the invention may need a preparation step shortly before consuming, e.g. the making of tea brew from tea-bags, leaf tea, herbal tea bags or herbal infusions or the solubilisation of powdered tea or powdered herbal tea. For these products it is preferred to adjust the level of isoflavones and calcium in the product such that one serving of the final product to be consumes has the desired levels of isoflavones and calcium as described above.

For ice-tea, ice herbal tea, carbonated ice tea, carbonated herbal infusions the typical size of one serving will be 250 ml or 250 grams. Preferred levels of isoflavone in these ready-to-drink products are 0.0004 to 0.1 wt %, more preferred, 0.0008 to 0.05 wt %, especially preferred 0.0016 to 0.016 wt %, most preferred, 0.002 to 0.006 wt %. Preferred levels of calcium in these ready to drink products are 0.04 to 0.8 wt %, more preferred, 0.045 to 0.4 wt %, especially preferred 0.05 to 0.2 wt %, most preferred, 0.05 to 0.08 wt %.

For products which are extracted to obtain the final product, generally the aim is to ensure that one serving of 250 ml or 250 grams comprises the desired amounts as indicated above. In this context it should be appreciated than normally only part of the isoflavones present in the tea based product to be extracted will eventually be extracted into the final tea drink. To compensate for this effect generally it is desirable to incorporate into the products to be extracted about 2 times the amount of isoflavones as is desired to have in the extract.

For leaf tea or tea-bags typically 1-5 grams of tea would be used to prepare a single serving of 250 mls. Preferred levels of isoflavone in the tea compound of such products would be 0.04 to 20 wt %, more preferred 0.08 to 10 wt %, especially preferred 0.16 to 5 wt%, most preferred 0.2 to 3 wt %.

Typically water soluble calcium salts would be used. Especially preferably is the use of calcium lactate which provides an advantageously good taste to the isoflavone containing tea. Preferred levels of calcium in the tea compound of such products would be 1-40 wt %, more preferred 1.5-20 wt %, especially preferred 2 to 15 wt %, most preferred 2.5 to 10 wt %.

If tea-bags are used, the isoflavone and calcium component may advantageously be incorporated into the tea component, however it will be appreciated that for some applications it may be advantageous to separate the isoflavone and/or the calcium from he tea, for example by incorporating them into a separate compartment of the tea bag or applying them onto the tea-bag paper.

### Salad Dressings or Mayonnaise

Generally dressings or mayonnaise are oil in water emulsions, The oil phase of the emulsion generally is 0 to 80 wt % of the product. For non fat reduced products the level of fat is typically from 60 to 80%, for salad dressings the level of fat is generally 10-60 wt %, more preferred 15-40 wt %, low or no fat dressings may for example contain triglyceride levels of 0, 5, 10, 15% by weight.

Dressings and mayonnaise are generally low pH products having a preferred pH of from 2-6.

Dressings or mayonnaise optionally may contain other ingredients such as emulsifiers (for example egg-yolk), stabilisers, acidifiers, biopolymers, bulking agents, flavors, coloring agents etc. The balance of the composition is water which could advantageously be present at a level of 0.1 to 99,9 wt %, more general 20-99 wt %, most preferred 50 to 98 wt %.

A typical size for an average serving of dressings or mayonnaise is 30 grams. Preferred isoflavone levels in the margarine or spread are 0.003 to 0.7 wt %, more preferred 0.007 to 0.33 wt %, especially preferred 0.003 to 0.13 wt %, most preferred 0.015 to 0.05 wt %. Preferred calcium levels are 0.3 to 7 wt %, more preferred 0.33 to 3.5 wt %, especially preferred 0.35 to 1.75 wt %, most preferred 0.35 to 0.7 wt %.

### EXAMPLE I

In the following examples the source of isoflavones is SoyLife as marketed by SoyLife Nederland B.V.

The composition of SoyLife is approximately as follows:

| Ingredient | wt % |
|---|---|
| Isoflavones¹ | 3% |
| Saponins | 4% |
| Protein | 40% |
| Fat | 11% |
| Fiber | 4% |
| Ash | 5% |
| Carbohydrates | 35% |
| Cholesterol | 0% |
| Tocopherols | 0.05% |
| α-Tocopherol | 0.008% |
| Lecithin | 2% |
| Water | balance |

¹glucosides

Novosoy obtained from Archer Daniels Midland may also be used.

The following vitamin mixes were used:

| | |
|---|---|
| Ingredient | wt % |

XR05837000 (ex Roche):

| | |
|---|---|
| Vitamin B6 | 2.9% |
| Vitamin B₁₂ | 7.8% |
| Vitamin E | 72% |
| Maltodextrin | balance |

GLATT PH990097:

| | |
|---|---|
| Calciumlactate | 73.8 |
| Vitamin B6 | 0.29% |
| Vitamin B12 | 0.78% |
| Vitamin E | 7.2% |
| Maltodextrin | balance |

### EXAMPLE II

Margarine or Other Spreads

A 40% fat spread is prepared by conventional spreads manufacturing. The composition of the product was is follows:

| Ingredients | wt % |
|---|---|
| CANOLA OIL | 30.6 |
| HYDROGENATED SOYBEAN OIL | 8.8 |
| LECITHIN | 0.2 |
| MYVEROL 1804, EMULSIFIER | 0.2 |
| FLAVOUR | 0.01 |
| GARLIC RESIN | 0.13 |
| VITA A/RED | 0.0066 |
| SALT | 1.50 |
| LACTIC ACID | 0.08 |
| POTASIUM SORBATE | 0.10 |
| EDTA | 0.0064 |
| PORK GELATIN | 2.0 |
| VITAMIN PREMIX, XR05837000 | 0.071 |
| SOYLIFE | 2.86 |
| TRICALCIUM PHOSPHATE | 1.88 |
| OREGANO LEAF | 0.50 |
| WATER | balance |

A typical serving of this product would be 14 grams, a suitable number of servings could be 5 to 30 per week, preferably 10-20.

If this spread is administered in effective amounts to human beings especially women aged 35-65 it is believed to result in advantageous health effects as described above.

### EXAMPLE III

A 40% fat spread is prepared by conventional spreads manufacturing. The composition of the product is as follows:

| Ingredient | wt % |
|---|---|
| SOYBEAN OIL | 30.7 |
| HYDROGENATED SOYBEAN OIL | 8.8 |
| LECITHIN | 0.22 |
| MYVEROL 1804, EMULSIFIER | 0.22 |
| FLAVOUR | 0.0125 |
| SALT | 1.5 |
| LACTIC ACID | 0.08 |
| POTASIUM SORBATE | 0.10 |
| EDTA | 0.0064 |
| PORK GELATIN | 2.0 |
| VITAMIN PREMIX, XR05837000 | 0.071 |
| SOYLIFE | 2.86 |
| TRICALCIUM PHOSPHATE | 1.88 |
| OREGANO LEAF | 0.50 |
| WATER | balance |

A typical serving size for this product would be 14 grams. Number of servings and advantageous health effects are as in Example I.

### EXAMPLE IV

Frozen Confectionery Product

The following ice-cream products are prepared by freezing in conventional ice-cream freezers.

| Description | wt % |
|---|---|
| Product A | |
| MILKFAT | 4.0% |
| NONFAT MILK | 14.0% |
| LIQUID SUCROSE (DRY WT) | 13.5% |
| LIQUID CORN 36 DE 80% | 7.75% |
| ENRICH 301 | 1.3% |
| STAR VITE A(25) 8.2#/ga | 0.0027% |
| 10/12 AMBER COCOA POWDER LB | 2.3% |
| NONFAT MILK | 0.015% |
| SoyLife | 0.50% |
| LIQUID SUGARED EGG YOLKS | 2.87% |
| WATER | balance |
| Product B | |
| MILKFAT PACKAGED | 4.5% |
| NONFAT MILK PACKAGED | 14.0% |
| LIQUID SUCROSE (DRY WT) | 14.0 |
| LIQUID CORN 36 DE 80% | 3.87% |
| ENRICH 301 | 1.3% |
| STAR VITE A(25) 8.2#/ga | 0.0034% |
| NONFAT MILK PACKAGED | 0.0166% |
| SoyLife | 0.55% |
| WATER | Balance |

### EXAMPLE V

Tea Based Products

Iced tea mix I

| Ingredient | Wt parts |
|---|---|
| MALTODEXTRIN | 37.1 |
| TEA POWDER | 8.7 |
| ASPARTAME | 2.6 |
| LEMON OIL POWDER | 0.95 |
| LEMON ESSENCE POWDER | 0.54 |
| MALIC ACID | 12.3 |
| OIL COATED MALIC ACID | 4.78 |
| MAGNESIUM OXIDE | 0.18 |
| SoyLife | 10.0 |
| VITAMIN PREMIX, =XR05837000 | 0.30 |
| CALCIUM LACTATE | 22.5 |

3.3 grams of the product can advantageously be used to prepare a serving of iced tea of 250 mls.

Iced tea mix II

| Ingredient | Wt parts |
|---|---|
| MALTODEXTRIN | 39.4 |
| TEA POWDER | 9.0 |
| ASPARTAME | 2.5 |
| PEACH FLAVOR | 3.6 |
| N&A APRICOT FLAVOR | 1.17 |
| CITRIC ACID | 9.05 |
| OIL COATED CITRIC ACID | 1.27 |
| MAGNESIUM OXIDE | 0.16 |
| SoyLife | 10.3 |
| VITAMIN PREMIX, =XR05837000 | 0.31 |
| CALCIUM LACTATE | 23.2 |

This mix can be used in the same way as mix I.

### EXAMPLE VI

Flavored tea bag A.

| Ingredient | Parts By Weight |
|---|---|
| SMOOTH BLEND TEA | 40.1 |
| HONEY FLAKES | 11.6 |
| LEMON FLAVOR | 4.0 |
| LEMON GRANULES | 3.80 |
| GLYCYRRHIZIN | 3.16 |
| HONEY GRANULE FLAVOR | 3.16 |
| SoyLife | 13.92 |
| VITAMIN/MINERAL PREMIX | 20.25 |
| PH990087 | |

4.74 grams of these are incorporated in a tea bag and used for the preparation of 250 mls of tea brew. About 50% of the soylife is extracted into the tea brew.

Herbal tea bag B.

| Ingredient | Parts By Weight |
|---|---|
| HIBISCUS | 7.3 |
| CHAMOMILE | 31.7 |
| CHICORY | 5.7 |
| PEACH FLAVOR GRANULES | 3.17 |
| APRICOT FLAVOR GRANULES | 0.63 |
| SoyLife | 20.95 |
| VITAMIN/MINERAL PREMIX | 30.48 |
| PH990087 | |

3.15 grams of these are incorporated in a tea bag and used for the preparation of 250 mls of tea brew. About 50% of the soylife is extracted into the tea brew.

Herbal tea bags C.

| Ingredient | Parts By Weight |
|---|---|
| ROSEHIPS | 9.6 |
| HIBISCUS | 26.4 |
| CHICORY ROOT | 7.21 |
| LICORICE ROOT | 0.72 |
| CITRIC ACID | 1.68 |
| ORANGE/PINEAPPLE/MANGO | 3.85 |
| ORANGE | 4.81 |
| ALMOND GRANULES | 0.96 |
| CINNAMON | 2.88 |
| ALLSPICE | 2.88 |
| SoyLife GRANULAR | 15.87 |
| VITAMIN/MINERAL PREMIX | 23.08 |
| PH990087 | |

4.16 grams of the tea is incorporated in a tea bag and used to prepare 250 mls of tea brew.

### EXAMPLE VII

EXAMPLE VII
Caesar Dressing
A dressing according to the following formulation is prepared.

| Ingredient | Wt Parts |
|---|---|
| DISTILLED WHITE VINEGAR | 2.0 |
| CANOLA OIL | 15.3 |
| SUCROSE | 7.5 |
| GRATED ROMANO CHEESE | 3.25 |
| SODIUM CHLORIDE GRANULAR | 2.2 |
| GARLIC POWDER | 3.0 |
| ANCHOVY PASTE | 1.5 |
| BLACK PEPPER | 0.5 |
| XANTHAN GUM | 0.27 |
| PROPYLENE GLYCOL ALGINATE | 0.10 |
| BALSAMIC VINEGAR | 3.07 |
| SOYLIFE** | 1.10 |
| VITAMIN PREMIX, ROCHE XR05837000 | 0.033 |
| GLUCONAL CALCIUM* | 335 |
| SODIUM BENZOATE GRANULAR | 0.09 |
| SORBIC ACID | 0.12 |
| EDTA | 0.007 |
| PHOSPHORIC ACID, 75% CONC. | 1.0 |
| POLYSORBATE 60 | 0.10 |
| CARAMEL POWDER | 0.04 |
| Water | To 100 |

*Calcium gluconate 80%/Calcium Lactate 20%
**Particle size 80% through 60 U.S. sieve size.

Italian dressing
An Italian dressing according to the following formulation is prepared:

| Ingredient | Parts By Weight |
|---|---|
| HIGH FRUCTOSE CORN SYRUP | 13.2 |
| CANOLA OIL | 15.3 |
| RED WINE VINEGAR | 1.4 |
| SODIUM CHLORIDE GRANULAR | 1.9 |
| PHOSPHORIC ACID, 75% CONC. | 1.0 |
| XANTHAN GUM | 0.25 |
| MINCED GARLIC | 0.91 |
| BLK PEPPER MED | 0.18 |
| RICE WINE VINEGAR, 10% | 6.85 |
| SODIUM BENZOATE GRANULAR | 0.085 |
| SORBIC ACID | 0.061 |
| SoyLife** | 1.1 |
| VITAMIN PREMIX, XR05837000 | 0.033 |
| GLUCONALCAL* | 3.35 |
| EDTA | 0.0066 |
| HERB DE PROVENCE | 0.19 |
| MINCED ONION | 0.19 |
| SUGAR | 2.25 |
| ANNATTO COLOR | 0.0047 |
| WATER | To 100 |

*Calcium gluconate 80%/Calcium Lactate 20%
**Particle size 80% through 60 U.S. sieve size

### EXAMPLE VIII

Four variations of ice cream flavors are prepared first by preparing a white mix and a chocolate mix as follows:

| | |
|---|---|
| Ingredient | % weight |

I. White Mix

| | |
|---|---|
| Milk fat | 4.50 |
| Non-fat milk solids | 15.25 |
| Liquid sugar | 14.00 |
| Liquid corn syrup | 3.88 |
| Stabilizer - guar/locust bean gum | 0.15 |
| Star vitamin A palmitate | 0.0034 |
| Roche vitamin mix: | 0.015 |
| Alpha-Tocopheryl acetate (vitamin E) | |
| Cyanocobalamin (vitamin B₁₂) | |
| Pyridoxine Hydrochloride (vitamin B₆) | |
| SoyLife (flour) | 0.56 |
| Water to 100% | ˜61.65 |

II. Chocolate Mix

| | |
|---|---|
| Milk fat | 4.00 |
| Non-fat milk solids | 15.25 |
| Liquid sugar | 13.47 |
| Liquid corn syrup | 7.75 |
| Stabilizer - guar/locust bean gum blend | 0.15 |
| Cocoa powder | 2.30 |
| SoyLife (flour) | 0.505 |
| Liquid sugared egg yolks | 2.87 |
| Star vitamin A palmitate | 0.0027 |
| Roche vitamin mix: | 0.0151 |
| Alpha-Tocopheryl acetate (vitamin E) | |
| Cyanocobalamin (vitamin B₁₂) | |
| Pyridoxine Hydrochloride (vitamin B₆) | |
| Water to 100% | ˜53.7 |

The Ice Cream Flavors then use the white or chocolate mix to prepare different flavors as follows:

| | Ingredients | % weight |
|---|---|---|
| A. French chocolate ice cream | Chocolate mix | 92.78 |
| | Milk chocolate flakes | 7.17 |
| | Vanilla flavor | 0.05 |
| B. Vanilla ice cream | White mix | 99.60 |
| | Vanilla flavor | 0.40 |
| C. Vanilla fudge ice cream | White mix | 90.74 |
| | Vanilla flavor | 0.364 |
| | Fat free liquid fudge variegate | 8.90 |
| D. Caramel praline ice cream | White mix | 82.35 |
| | Vanilla flavor | 0.40 |
| | Liquid caramel variegate | 11.65 |
| | Praline nuts & toffee | 5.60 |
| | (particulate) | |

### EXAMPLE IX

EXAMPLE IX
A tea formula similar to Example VI C. is compared to a standard.

| | Control | | Isoflavone | |
|---|---|---|---|---|
| Rosehips | 1.8 | | 1.2 | |
| Hibiscus | 3.0 | | 3.48 | |
| Chicory | 1.0 | | 0.9 | |
| Licorice | .09 | | .09 | |
| Citric acid | .18 | | .21 | |
| Orange and mango flavor | .84 | | .86 | |
| Orange flavor | .36 | | .60 | |
| Almond flavor | — | | .12 | |
| Cinnamon | — | | .36 | |
| Allspice | — | | .36 | |
| SoyLife granular | 1.98 | | 1.98 | |
| Vitamins* | 2.88 | | 2.88 | |
| | 12.13 | g | 13.04 | g |

*Glatt/PH990097

* Glatt/PH990097

Testing is conducted in-house with 50 employee users of herbal tea who are acceptors of citrus flavor. Samples are evaluated hot. Panelists rate each sample on a 9 point hedonic scale for overall acceptability and select a preferred sample. No significant acceptability differences are detected at the 95% confidence level (CL). Both samples are rated between “Like Slightly” and “Like Moderately”. At the 90% CL, the isoflavone prototype is preferred over current production Tea.

### Sensory Methods

Sensory Approach

An in-house acceptability and preference test is conducted with a panel of 50 employee users of herbal tea who are acceptors of citrus flavor. Panelists are requested to rate each sample on a 9 point hedonic scale and select a preferred sample.

Sample Presentation

Samples are brewed for 5 minutes and served at approximately 150° F., in labeled white Corelle cups, under white lights in the Sensory booths, using balanced orders of presentation. Samples are identified to panelists as Herbal tea.

Results

No significant acceptability differences are found between samples at the 95% Confidence level (CL). At the 90% CL, the isoflavone prototype containing some additional flavors is preferred over current production tea.

TABLE I
Current Product vs. Isoflavone Containing Prototype
Acceptability/Preference
(N = 50)

| | Mean Rating | Number | |
|---|---|---|---|
| Sample | Overall Liking | Preferring | % |
| Project Eve Prototype | 6.6 | 31 | 62 |
| Citrus Almond Spice | | | |
| Benchmark sample | 6.5 | 19 | 38 |
| Current Citrus Blossom | | | |

Acceptability Rating Scale

| | | | | | | | | |
|---|---|---|---|---|---|---|---|---|
| 1 | 2 | 3 | 4 | 5 | 6 | 7 | 8 | 9 |
| Dislike | Dislike | Dislike | Dislike | Neither Like | Like | Like | Like | Like |
| Extremely | Very Much | Moderately | Slightly | nor Dislike | Slightly | Moderately | Very Much | Extremely |

### EXAMPLE X

EXAMPLE X
The tea formula of Example VI A. is compared to a standard.

| | Control | | VI A. | |
|---|---|---|---|---|
| Smooth blend tea | 1.9 | | 1.9 | |
| Honey flakes | .55 | | .55 | |
| Lemon flavor | — | | .19 | |
| Lemon granules | .18 | | .18 | |
| Glycyrrhizin | .15 | | .15 | |
| Honey granules flavor | — | | .16 | |
| SoyLife | .66 | | .66 | |
| Vitamin pre mix* | .96 | | .96 | |
| | 4.40 | g | 4.75 | g |

*Glatt PH990097
Calcium lactate 0.74
Vitamin mix 0.1

Testing is conducted in-house with 50 employee users of flavored tea. Samples are evaluated hot. Panelists rate each sample on a 9 point hedonic scale for overall acceptability and select a preferred sample. No significant acceptability differences are detected. Both samples are rated slightly above “Like Slightly”. However, the isoflavone containing prototype was significantly preferred over current Honey & Lemon (p=0.05)

### Sensory Methods

Sensory Approach

An in-house acceptability and preference test is conducted with a panel of 50 employee users of flavored tea who are acceptors of honey and lemon flavors. Panelists are requested to rate each sample on a 9 point hedonic scale and select a preferred sample.

Sample Presentation

Samples are brewed for 3 minutes, and served at approximately 150° F., in labeled white Corelle cups under white lights in the Sensory booths, using balanced orders of presentation.

Results

No significant acceptability differences are found between samples, although the isoflavone sample contained more flavoring. The isoflavone prototype was significantly preferred over current production Honey & Lemon Flavored Tea.

TABLE I
Current Product vs. Isoflavone Containing Prototype
Acceptability/Preference
(N = 50)

| | Mean Rating | Number | |
|---|---|---|---|
| Sample | Overall Liking | Preferring | % |
| Project Eve Prototype | 6.3 | 32a* | 64 |
| Honey & Lemon | | | |
| Benchmark sample | 6.3 | 18b | 36 |
| Current Honey & Lemon | | | |

*Values followed by a different letter are significantly different at p = 0.05.

Acceptability Rating Scale

| | | | | | | | | |
|---|---|---|---|---|---|---|---|---|
| 1 | 2 | 3 | 4 | 5 | 6 | 7 | 8 | 9 |
| Dislike | Dislike | Dislike | Dislike | Neither Like | Like | Like | Like | Like |
| Extremely | Very Much | Moderately | Slightly | nor Dislike | Slightly | Moderately | Very Much | Extremely |

### EXAMPLE XI

Examples of typical weekly amounts of products of the invention, preferably administered to women of 35-65 years.

Weekly diet:

| | | |
|---|---|---|
| A. | 50 | grams of spread of example III pIus 5 servings of tea mix |
| | | according to example V |
| B. | 500 | grams of ice-cream according to example IV |
| C. | 75 | grams of the spread according to example II |
| D. | 10 | tea bags according to example VI |
| E. | 5 | servings of dressing of example VII plus 250 grams of |
| | | ice-cream |

Other variations and combinations are possible, each providing within a normal diet an advantageous amount of isoflavones and calcium.

It is understood that the examples and embodiments described herein are for illustrative purposes only and that various modifications or changes in the light thereof will be suggested to persons skilled in the art and are to be included within the spirit and purview of this application and the scope of the appended claims.

1. A food product comprising isoflavones and calcium, wherein the weight ratio of isoflavones to calcium is from 1 to 2 to 1 to 50.

2. Food product according to claim 1, being part of the normal daily diet.

3. Food product according to claim 1 wherein the food product is selected from the group consisting of margarines or other spreads, tea based drinks, dressings, mayonnaise and frozen confectionery products.

4. Food product according to claim 1, wherein the isoflavones have been obtained from soy.

5. Food product according to claim 1, wherein calcium has been added to the product as an edible salt, selected from the group consisting of calcium maleate, calcium tartrate, calcium gluconate, calcium lactate, calcium acetate, calcium carbonate and calcium phosphate.

6. Food product according to claim 1, wherein the weight ratio of isoflavone to calcium is from 1 to 3 to 1 to 30.

7. Food product according to claim 1, wherein the level of isoflavone is chosen such that a normal serving of the food product comprises 1 to 200 milligrams of isoflavone.

8. Food product according to claim 1, wherein the level of calcium is chosen such a normal serving of the food product comprises 100 to 2000 milligram of calcium.

9. Food product according to claim 1, which is a margarine or other spread, wherein the isoflavone level is 0.007 to 1.5 wt %, and the calcium level is 0.7 to 15 wt %.

10. Food product according to claim 1, which is a frozen confectionery product having an isoflavone level of 0.0015 to 0.3 wt and a calcium level of 0.15 to 3 wt %.

11. Food product according to claim 1, which is a tea based beverage wherein the isoflavone level is 0.0004 to 0.1 wt % and the calcium level is 0.04 to 0.8 wt %.

12. Food product according to claim 1 which is a tea-leaf or tea-bag product comprising leaf tea wherein the isoflavone level is 0.04 to 20 wt % and the calcium level is 1 to 40 wt % based on the weight of the tea compound present in the tea-leaf or tea-bag.

13. Food product according to claim 1, which is a dressing or mayonnaise having an isoflavone level 0.003 to 0.7 wt %, and a calcium level of 0.3 to 7 wt.

14. A method for the prevention or reduction of symptoms of osteoporosis and/or menopausal syndrome and/or hot flushes and/or cardiovascular disease and/or breast cancer and/or migraine which comprises administering to a subject an effective amount of a food product according to claim 1.

15. A method according to claim 14, wherein the subject is a female aged 35 to 65 years.

16. A method according to claim 13, wherein the amounts and types of products are chosen that per week on average 20 to 1000 mg of isoflavones is consumed via products according to claim 1.

17. A Use of isoflavones and calcium in a weight ratio of isoflavones to calcium from 1 to 2 to 1 to 50 in the preparation of a food product for the prevention or reduction of symptoms of osteoporosis and/or menopausal syndrome and/or hot flushes and/or cardiovascular disease and/or breast cancer and/or migraine.

18. Use according to claim 17, wherein the food product is intended to be administered to females of 35 to 65 years.

19. Use according to claim 17, wherein the food product is selected from the group consisting of margarines or other spreads, tea based drinks, dressings, mayonnaise and frozen confectionery products.
